(19) United States
(12) Patent Application Publication
(10) Pub. No.: US 2020/0121227 A1
(43) Pub. Date: Apr. 23, 2020

(54) SYSTEM AND METHOD FOR QUANTIFYING JOINT CHARACTERISTICS

(71) Applicant: ARTHREX, INC., Naples, FL (US)

(72) Inventors: Arley Perez, III, Naples, FL (US); Thomas Edward Anstead, Naples, FL (US); Ryosuke Kuroda, Nishinomiya (JP); Kouki Nagamune, Fukui (JP)

(21) Appl. No.: 16/336,834

(22) PCT Filed: Sep. 21, 2017
(86) PCT No.: PCT/US17/52801

§ 371 Date: Mar. 26, 2019

Related U.S. Application Data
(60) Provisional application No. 62/400,518, filed on Sep. 27, 2016.

(51) Int. Cl. A61B 5/11; A61B 5/05; A61B 5/00; G06F 3/0354

(52) U.S. Cl. CPC A61B 5/1121; A61B 5/05; A61B 5/7475; A61B 2562/0223; A61B 5/6828; G06F 3/03545; A61B 5/742

(57) ABSTRACT

Systems, devices, methods, and software for measuring and quantifying a limb movement are disclosed. An electromagnetic field generator generates an electromagnetic field; a plurality of electromagnetic sensors, positionable inside of the electromagnetic field and at different locations on a limb, generate position and orientation data; an electromagnetic stylus positionable inside of the electromagnetic field generates position and orientation data when activated; a processor coupled to the plurality of electromagnetic sensors and the stylus receives the data generated by the sensors and the stylus and calculates an angular movement of the limb and translation of an appendage coupled to a joint; and a display coupled to the processor displays at least one of the calculated angular movement and translation.

### CROSS-REFERENCE TO RELATED APPLICATIONS

This application is a national phase entry application of PCT Application No. PCT/US2017/052801 filed on Sep. 21, 2017, entitled SYSTEM AND METHOD FOR QUANTIFYING JOINT CHARACTERISTICS, which claims priority of U.S. Provisional Patent Application No. 62/400,518, entitled JOINT STABILITY QUANTIFICATION SYSTEM AND METHOD, filed on Sep. 27, 2016, the entire contents of both of which are hereby incorporated herein by reference.

### BACKGROUND

The present disclosure relates to orthopedic measuring devices and methods, and, more particularly, to devices and methods for measuring and quantifying limb movements such as clinical knee maneuvers.

### SUMMARY

The present invention is directed to devices, systems, methods, and software for measuring, recording, processing and quantifying limb movement(s), including various clinical knee maneuvers.

The devices, systems, methods, and software for measuring, recording, processing and quantification of limb movement, including various clinical knee maneuvers, are independent of a person performing such tests. The devices, systems, methods, and software detailed below provide a way to record and store the results of the tests, to compare the results of the tests with the test results of other patients or other limbs of a same patient, and/or to compare the results of the tests with test results from a different date.

The present invention, according to an exemplary embodiment, is directed to a system for quantifying joint characteristics, comprising: an electromagnetic field generator configured to generate an electromagnetic field; a plurality of electromagnetic sensors positionable inside of the electromagnetic field, wherein each sensor is positionable at a different location on a limb and wherein each sensor is configured to generate position and orientation data; an electromagnetic stylus positionable inside of the electromagnetic field, wherein the stylus is configured to generate position and orientation data when activated; a processor coupled to the plurality of electromagnetic sensors and the stylus and configured to receive the data generated by the plurality of sensors and the stylus, wherein the processor is further configured to calculate at least one of an angular movement of the limb and a translation of an appendage coupled to a joint, wherein the limb comprises the appendage and the joint; and a display coupled to the processor and configured to display at least one of the calculated angular movement and translation.

In another exemplary embodiment, the system is further configured to display movement of the limb based on the data generated by the plurality of sensors. In an additional exemplary embodiment, the plurality of sensors are configured to measure movement of the limb while at least one of a Lachman, Dial, Varus/Valgus, and Pivot-Shift test is conducted on the limb. In an additional exemplary embodiment, the processor further comprises a memory configured to store at least one of the calculated angular movement and translation per test conducted on the limb.

In yet another exemplary embodiment, at least one of the plurality of sensors is removably attachable to a bone of the limb with at least one of a pin, screw and fixture. In another exemplary embodiment, at least one of the plurality of sensors is removably attachable to the limb with at least one of a strap, tape and an adhesive.

Optionally, the system is configured to display the calculated angular movement and translation of at least one of a left limb and a right limb. Optionally, the system is configured to simultaneously display the calculated angular movement and translation of left and right limbs. The joint may comprise a knee joint, and the appendage may comprise a femur and a tibia. The measured translation may be anterior-posterior translation.

In another exemplary embodiment, the system is configured to further display a first vertical line in conjunction with a peak of the displayed translation versus time and a second vertical line in conjunction with a low of the translation displayed versus time, and wherein the translation is calculated during a Pivot-Shift test conducted on the limb. Optionally, the processor is configured to further calculate a Pivot-Shift reduction between the first and second vertical lines. Optionally, the processor is configured to further calculate a distance (PSr) between two peaks of the translation displayed while a Pivot-Shift test is conducted on the limb. Optionally, the processor is configured to further calculate at least one of a velocity (PSv) by dividing the distance (PSr) by a sampling frequency and an acceleration (PSa) by dividing a difference between the calculated velocities by the sampling frequency. The system may be configured to further display at least one of the velocity (PSv) versus time and acceleration (PSa) versus time. The processor may further comprise a memory configured to store the calculated velocity and acceleration information per limb on which the Pivot-Shift test is conducted.

The present invention, according to an additional exemplary embodiment, is directed to a method for quantifying joint characteristics comprising: generating an electromagnetic field around a limb; placing a plurality of electromagnetic sensors at different locations on the limb; measuring an initial location of a plurality of landmarks of the limb using an electromagnetic stylus; measuring movement of the plurality of electromagnetic sensors; calculating at least one of an angular movement of the limb and a translation of an appendage coupled to a joint using the initial locations of the plurality of landmarks of the limb and measured movement of the plurality of electromagnetic sensors, wherein the limb comprises the appendage and the joint; and displaying at least one of the calculated angular movement and translation.

Optionally, the method further comprises determining a location of the limb using the initial locations of the plurality of landmarks of the limb and measured movement of the plurality of electromagnetic sensors; and displaying an image of the limb based on the determined location. Optionally, the method further comprising displaying a movement of the limb based on the measured movement of the sensors.

In an additional exemplary embodiment, the step of measuring movement comprises measuring the movement of the limb while at least one of Lachman, Dial, Varus/Valgus, and Pivot-Shift tests are conducted on the limb. Optionally, the method further comprises recording the measured movement of the limb during the test; and storing the angular movement and translation information per limb. Optionally, the stored angular movement and translation information of left and right limbs is displayed on a same screen.

In an additional exemplary embodiment, the method further comprises: displaying a first vertical line in conjunction with a peak of the translation displayed versus time and a second vertical line in conjunction with a bottom of the translation displayed versus time, wherein the translation is calculated while a Pivot-Shift test is conducted on the limb. Optionally, the method further comprises calculating a Pivot-Shift reduction between the two vertical lines. Optionally, the method further comprises calculating distance (PSr) between two peaks of the translation displayed while a Pivot-Shift test is conducted on the limb. Optionally, the method further comprises calculating at least one of a velocity (PSv) by dividing the distance (PSr) by a sampling frequency and an acceleration (PSa) by dividing a difference between the calculated velocities by the sampling frequency. Optionally, the method further comprises displaying at least one of a velocity (PSv) versus time and an acceleration (PSa) versus time. Optionally, the method further comprises storing the at least one of velocity (PSv) and acceleration (PSa) information per limb on which the Pivot-Shirt test is conducted.

In an additional exemplary embodiment, the method further comprises: prompting a user to enter a plurality of landmarks of the limb using the stylus; and receiving location and orientation data for the plurality of landmarks of the limb. Optionally, the step of prompting the user to enter a plurality of landmarks further comprises graphically prompting the user to enter location information for at least one of a greater trochanter, medial epicondyle, lateral epicondyle, intersection of the knee joint line and medial collateral ligament, fibula head, medial malleolus and a lateral malleolus.

### BRIEF DESCRIPTION OF THE DRAWINGS

The features, aspects and advantages of the present invention will become better understood with regard to the following description, appended claims and accompanying figures wherein:

FIG. 1 illustrates a system for measuring and quantifying limb movement according to an embodiment.

FIG. 2 illustrates a close up view of a portion of the system of FIG. 1.

FIG. 3 illustrates sensors attached to a limb for use with the system of FIG. 1.

FIG. 4 is a flowchart of a process for measuring limb movement and quantifying the movement according to an exemplary embodiment.

FIG. 5 illustrates a display screen showing a start page of a system measuring and quantifying limb movement according to an exemplary embodiment.

FIG. 6 illustrates a display screen guiding a user to select a leg of which a movement will be measured and quantified according to an exemplary embodiment.

FIGS. 7-9 illustrate a display screen guiding a user to landmark a part of a leg according to an exemplary embodiment.

FIG. 10 illustrates a display screen showing the signal strength of a stylus and sensors used to measure a movement of a limb according to an exemplary embodiment.

FIG. 11 illustrates a display screen showing types of tests to be performed on a leg of which a movement will be measured and quantified according to an exemplary embodiment.

FIGS. 12-14 illustrate a display screen showing a limb and its movement according to an exemplary embodiment.

FIGS. 15 and 16 illustrate a display screen showing the recording and playing a limb movement measured and quantified according to an exemplary embodiment.

FIG. 17 illustrates a display screen showing a flexion degree and translation plotted against time based on a limb movement measured and quantified according to an exemplary embodiment.

FIG. 18 illustrates a display screen from which a user can select at least one of the acceleration, translation, velocity, flexion degree, internal/external rotation, and varus/valgus information of a limb to be displayed according to an exemplary embodiment.

FIGS. 19 and 20 illustrate a display screen from which a user can select stored limb movement information so that the stored information is displayed as a mean of comparison with other limb movement information according to an exemplary embodiment.

### DETAILED DESCRIPTION

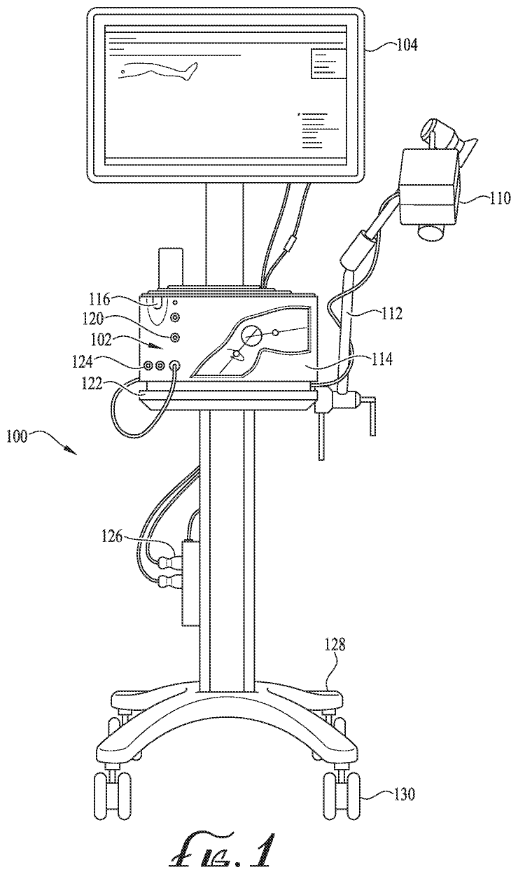

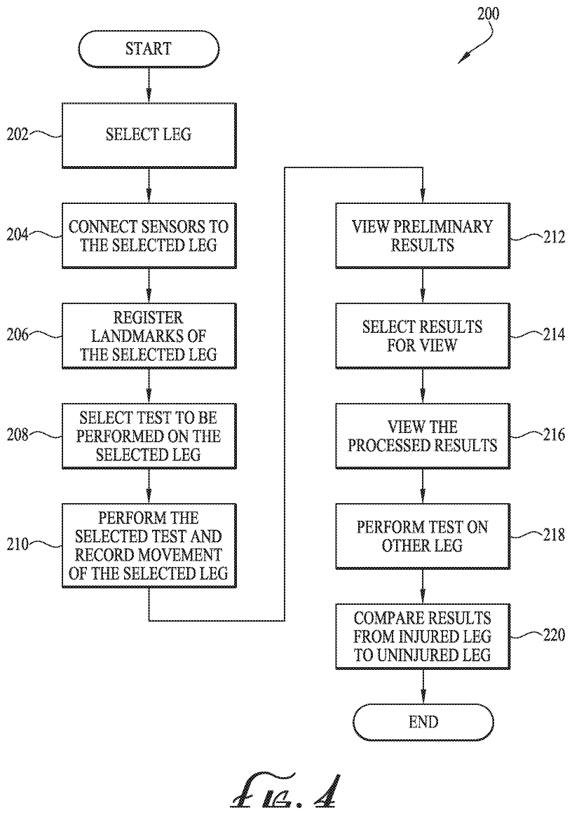

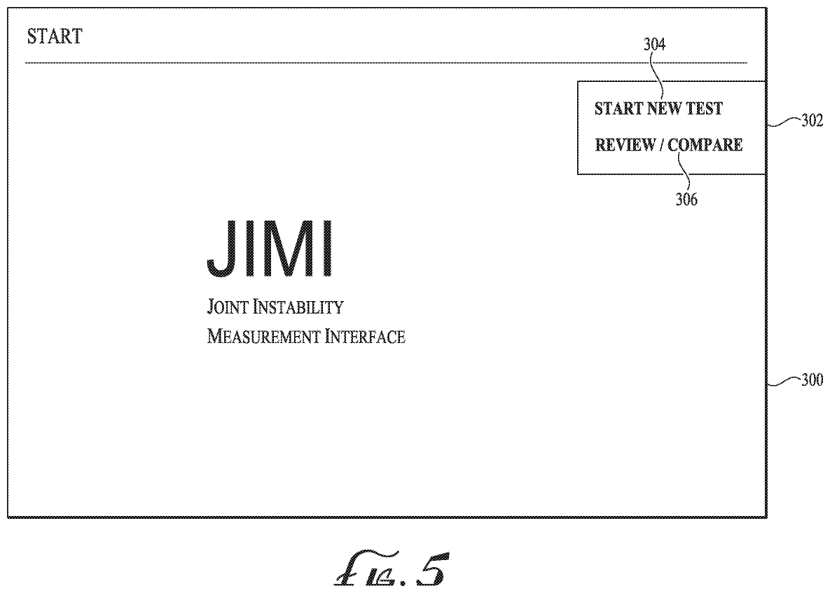

An exemplary system 100 according to an embodiment of the present disclosure is depicted in FIGS. 1 to 3. System 100 comprises a processor 102 and a display 104. The processor 102 has a microprocessor operatively coupled to a memory system. The memory system may include one or more memory devices (RAM, ROM, disk or other storage format). The memory system stores software executed by the processor to implement process 200 shown in FIG. 4. The display 104 is attached to processor 102 and is discussed further with regard to FIGS. 5-20.

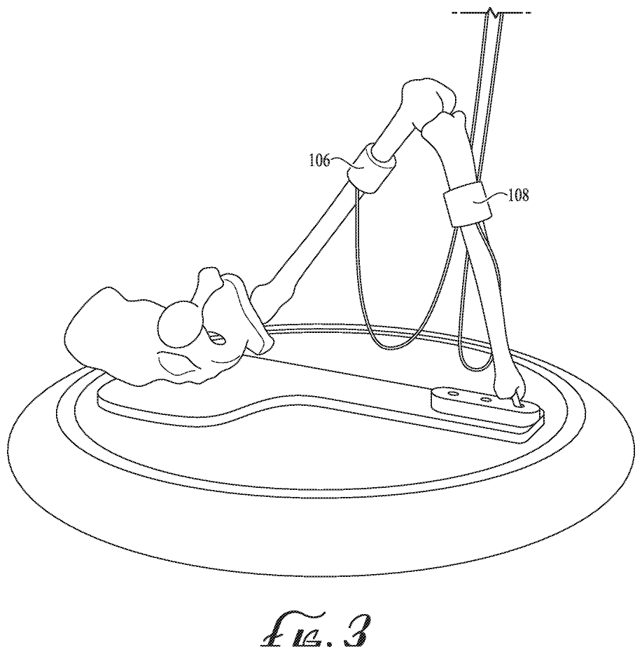

In system 100, processor 102 is in communication with two electromagnetic sensors: a femoral sensor 106 and tibial sensor 108 as illustrated in FIG. 3. The sensors 106, 108 are attached via a strap around the skin of a patient. Alternatively, the sensors 106, 108 are attached using tape or an adhesive. Alternatively, the sensors 106, 108 can be attached via pins inserted in bones. Alternatively, the sensors 106, 108 can be attached to a bone using a screw or a fixture.

The sensors 106, 108, when attached to a limb of a patient, measure the movement of the limb. For instance, as illustrated in FIG. 3, sensors 106, 108 may be attached to a leg of a patient. Sensors 106, 108 generate data based on measured movement, and transfer the generated data to the processor 102. According to an embodiment, the processor 102, utilizes software stored in memory and executed by a processor to calculate an angular movement (such as a flexion degree) of a leg and translation of a tibia in relation to a femur based on the data received from the sensors 106, 108. The processor 102 may output the flexion degree and translation versus time to the display 104, as described below.

An electromagnetic field generator (EMFG) 110 generates an electromagnetic field. In response to the electromagnetic field generated by the EMFG 110, sensors 106, 108 can communicate their position and orientation in relation to the EMFG 110 to the processor 102. When an electromagnetic sensor is used to measure the movement of a limb, the EMFG 110 may act as a global coordinate system for the system 100. The system may utilize known electromagnetic field generators and sensors, such as those from Polhemus in Colchester, Vt.

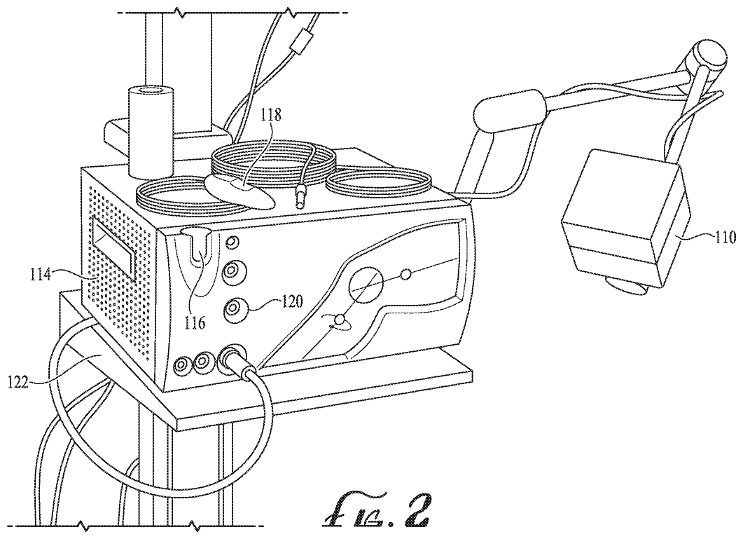

FIGS. 1 and 2 illustrate the front and rear view of the system 100. According to an illustrative embodiment, EMFG 110 is attached to a boom 112.

An enclosure 114 surrounding the processor 102 may possess a stylus housing 116 for a stylus 118. As with the sensors 106, 108, the stylus 118 can communicate its position and orientation in relation to the EMFG 110 to the processor 102. The system may utilize a stylus 118 from Polhemus in Colchester, Vt.

The user of the system 100 may use the stylus 118 to landmark a part of a limb. The processor 102 may use the landmark to calculate movement information of a limb, such as flexion degree of a leg and translation of a tibia in relation to a femur. The landmark and display process are described in greater detail below.

As illustrated in FIGS. 1 and 2, enclosure 114 also may include connectors 120 for interfacing with sensors 106, 108 and with stylus 118. Through the connectors 120, processor 102 may communicate with the sensors 106, 108 and with the stylus 118. Various types of wired or wireless communication methods known in the art may be implemented for communication between the processor 102 and the sensors 106, 108 and/or the stylus 118.

The boom 112 also allows the EMFG 110 to be placed within appropriate proximity of a patient on whom the sensors 106, 108 are attached. According to an embodiment, the display 104 is a touch panel that may be used to communicate user input to the processor 102. The processor 102 is loaded with a system software and allows the user to interface with the software via the touch panel. The software also provides relevant information to the user. The software of the processor 102 can be executed on a processor of the processor 102 to calculate the relevant information, e.g., flexion degree and translation. The display 104, may display the information calculated by the processor of the processor 102. In an additional embodiment, the processor 102 may be an integrated touch panel with processor.

The enclosure 114 is mounted to a shelf 122. The enclosure 114 has peripheral ports for attaching peripheral components to the processor 102. The enclosure 114 protects the processor 102 from fluids. The processor 102 is connected to a power supply 126 that accepts 110/240 V.

Caster booms 128 of FIG. 1 enhance the stability of the system and provide a location for mounting casters 130.

FIG. 4 is a flowchart showing process 200 using system 100 to measure movement in a limb and quantify the measured movement according to an exemplary embodiment. First, either a right or a left leg of a patient is selected for measurement in step 202. Second, the selected leg of a patient is placed in the desired testing position and sensors 106, 108 are attached to the selected leg in step 204.

Next, in step 206, the user registers landmarks of the limb. The user may use stylus 118 as described below to landmark the limb. In step 208, the user selects a test to be conducted on the selected leg. As explained below, the display 104 prompts the user to select (from a set of tests) the test the user plans to perform on the limb. For instance, the user may select a Pivot-Shift test among the set of tests displayed on the display 104.

After the selection of the test, the user performs the test and the movement of the leg is recorded while being displayed on the display 104 in real time in step 210. The processor 102 may also calculate the angular movement of a leg and translation of a tibia in relation to a femur and display preliminary results for the user to view on the display 104 in step 212.

The user may also select different types of information to be displayed on the display 104 in step 214. For instance, when a Pivot-Shift test is performed on a leg, a processor of PC 6 can calculate the velocity and acceleration of the leg movement based on the preliminary results as described below. The user may select that the velocity and acceleration are plotted against time and the calculated velocity and acceleration plotted against time are then displayed on the display 104 in step 216.

After a test is performed on a leg, the user may perform the test on the other leg of the patient in step 218. The test of the other leg will result in display of the movement of the other leg and the relevant preliminary results on the display 104. A comparison of the test results of the first leg and the second leg may be displayed on the display 104 in step 220.

It should be understood that process 200 as described above is not limiting. Additional steps may be performed at any time during process 200 or the described steps may be altered or not performed, or the steps may not be performed in the sequence outlined above.

FIG. 5 illustrates a display screen showing a start page of a system measuring and quantifying limb movement according to an exemplary embodiment. Upon executing the software loaded on processor, a start page screen 300 is displayed on the display 104. The software may be executed by starting an application corresponding to the software through a web browser, such as Google Chrome.

Start page screen 300 shows a software navigation box 302. Navigation box 302 is used to navigate the software and provides varying options that are dependent upon the screen the user is on. As shown in FIG. 5, the navigation box 302 on the start screen may prompt a user to select “Start New Test” 304 or “Review/Compare” existing results 306.

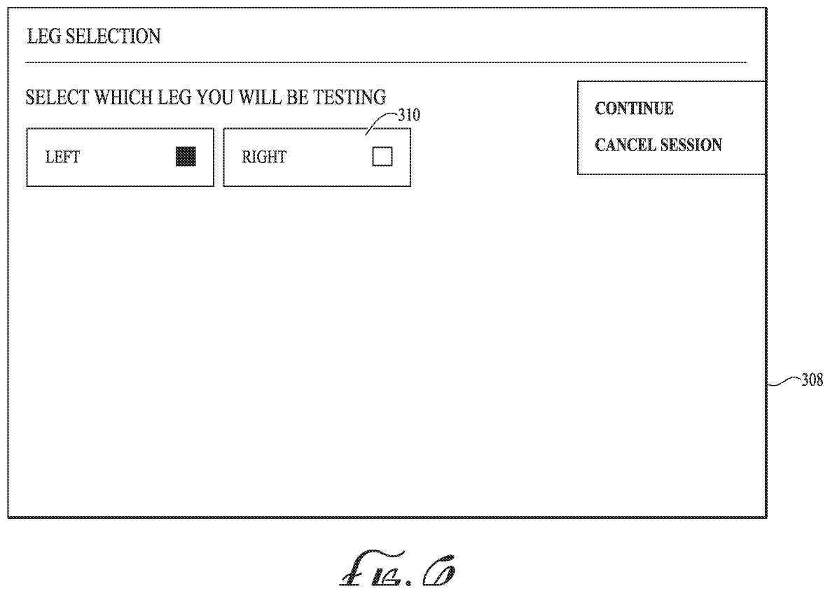

FIG. 6 illustrates a display screen guiding a user to select a leg of which a movement will be measured and quantified according to an exemplary embodiment. Leg selection screen 308 presents the user with leg buttons 310 that prompt the user to select the leg that will be tested.

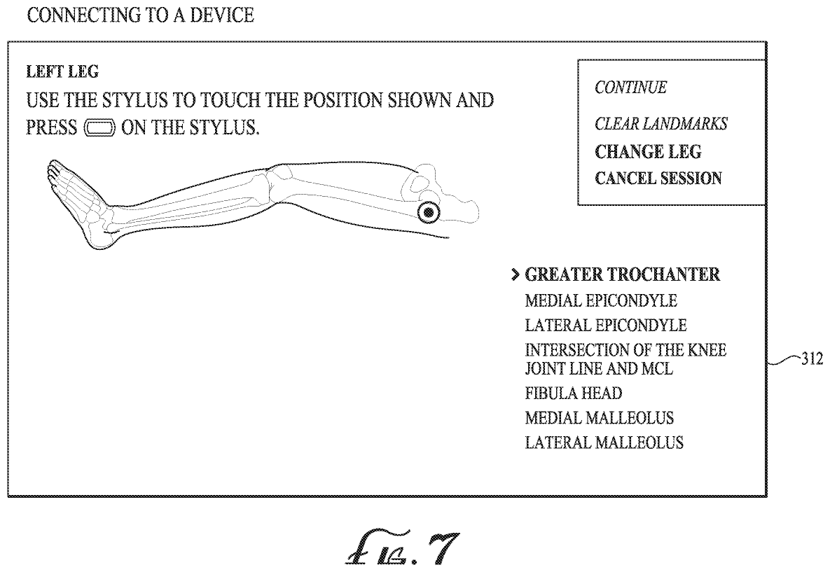

FIGS. 7, 8, and 9 illustrate a display screen guiding a user to landmark parts of a leg according to an exemplary embodiment. Upon selection of a leg to perform a test, the display of PC 6 displays connection screen 312 of FIG. 7. The connection screen 312 indicates that the software is connecting to the electromagnetic electronics. If there is a connection error, the connection screen 312 will inform the user.

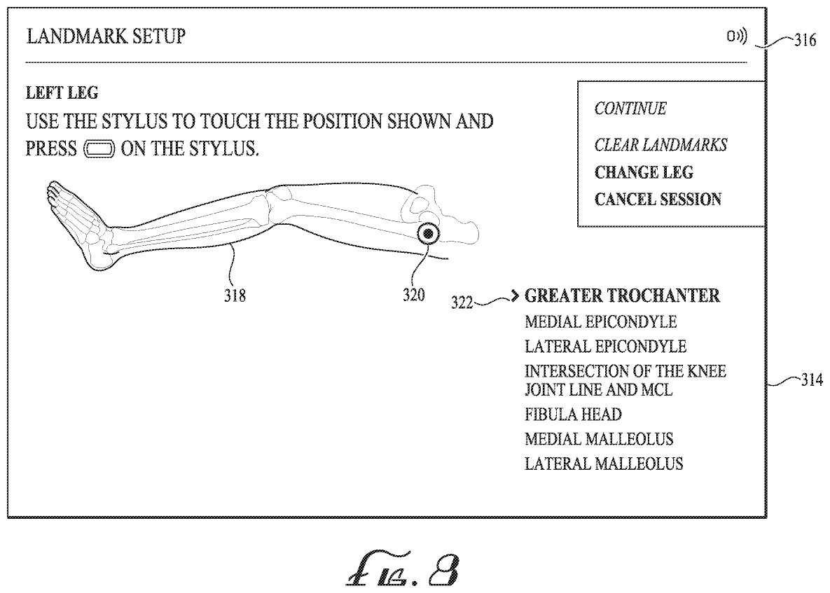

Upon successful connection with the electromagnetic electronics, the display 104 displays landmark setup screen 314 as illustrated in FIG. 8. The landmark setup screen 314 displays the signal strength 316, leg representation 318, landmark indicator 320, and landmark list 322. The leg representation 318 provides an overall view of the leg depending on which leg was selected. Landmark indicator 320 provides an indication of the physical location of the current landmark required. The landmarks are selected by pressing a button on the stylus 118 or depressing a footswitch (not shown). In an embodiment, the footswitch emulates the button on the stylus to submit to the processor 102 the location and orientation of the stylus at the time the footswitch is depressed. The Landmark list 322 provides further indication of the landmark that must be identified.

In an embodiment, one sensor 106 is positioned on a patient's femur approximately 10 cm proximal to the knee joint line and another sensor 108 is positioned to the tibia approximately 7 cm distal to the knee joint line. The landmarks are entered to allow software executing on processor 102 to calculate limb information with reference to the initial location and orientation of the sensors 106, 108 and changes to the location and orientation of the sensors. By securely fastening the sensors 106, 108 to the limb and establishing initial landmark locations, the system allows for calculations relating to a knee joint line without sensors actually being positioned on the knee joint line.

In an embodiment, seven different landmarks are entered into the system; three for the femur and four for the tibia. In another embodiment, at least one additional sensor is used and entry of at least one less landmark is necessary. In another embodiment, the number of entered landmarks depends on the test being conducted. In an embodiment, landmarks are selected from a greater trochanter, medial epicondyle, lateral epicondyle, intersection of the knee joint line and medial collateral ligament, fibula head, medial malleolus and lateral malleolus.

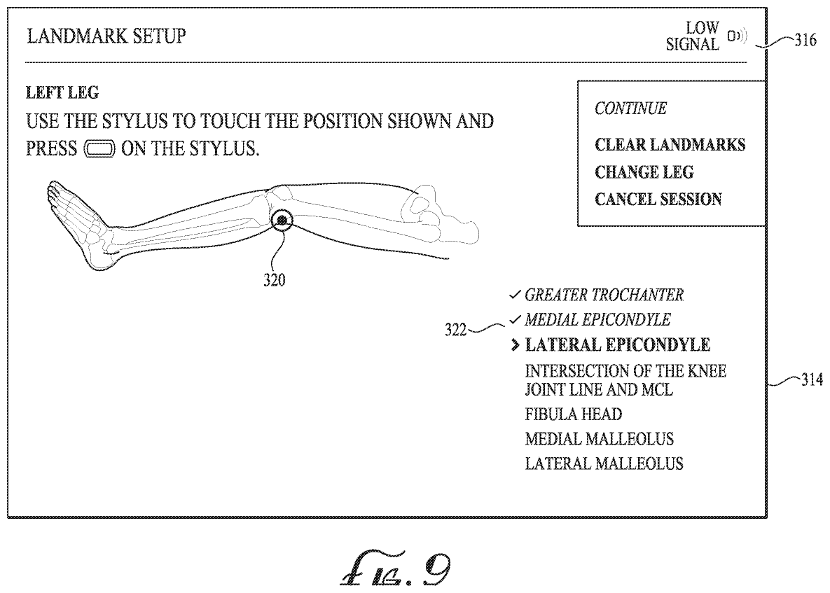

The landmark list 322, as illustrated in FIG. 9, shows the third landmark pending selection. In an embodiment, identified landmarks are indicated with a check beside them, a current landmark is indicated with a right chevron, and future landmarks are shaded. If a mistake is made, the user may re-indicate a specific landmark by tapping on the desired landmark and then indicating the landmark with the stylus as discussed above.

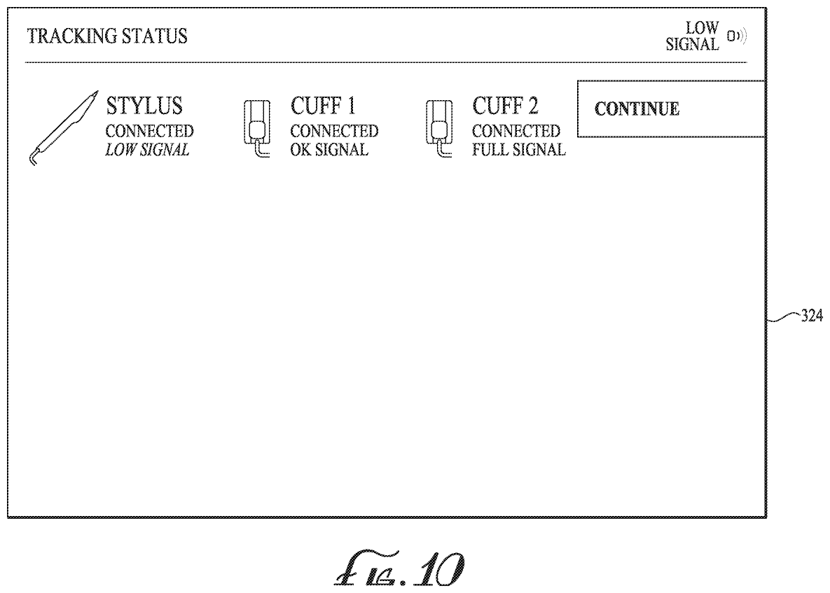

FIG. 10 illustrates a signal strength display screen 324 showing a signal strength of the stylus 118 and the sensors 106, 108 used to measure movement of the limb according to an exemplary embodiment. When a user taps on signal strength indicator 316 of landmark setup screen 314, tracking status screen 324 is shown on the display 104. The signal strength display screen 324 displays the signal strength of each electromagnetic peripheral.

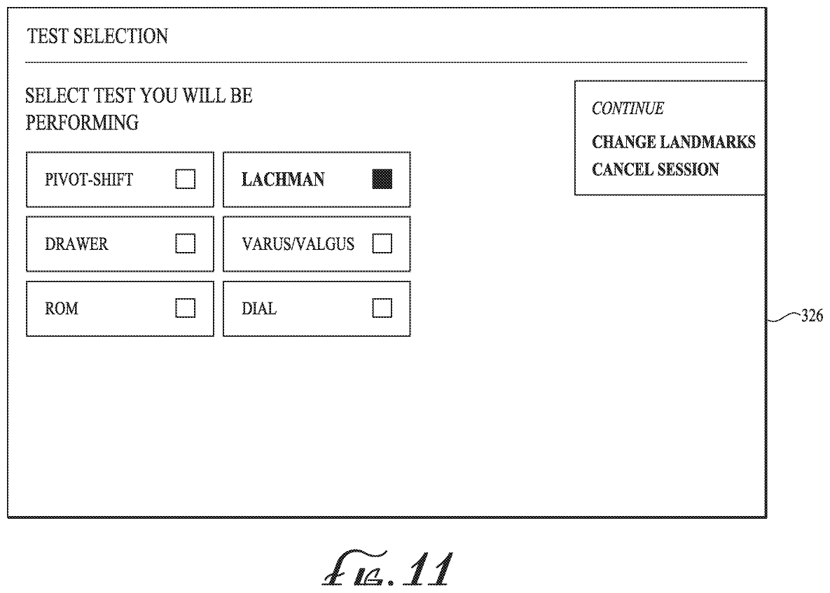

FIG. 11 illustrates a test selection screen 326 showing types of tests to be performed on a leg of which a movement will be measured and quantified according to an exemplary embodiment. After the last landmark is successfully registered, the user may proceed to the test selection screen 326 as illustrated in FIG. 11. According to an exemplary embodiment, the test selection screen 326 provides six different test options: Pivot-Shift, Lachman, Drawer, Varus/Valgus, ROM and Dial.

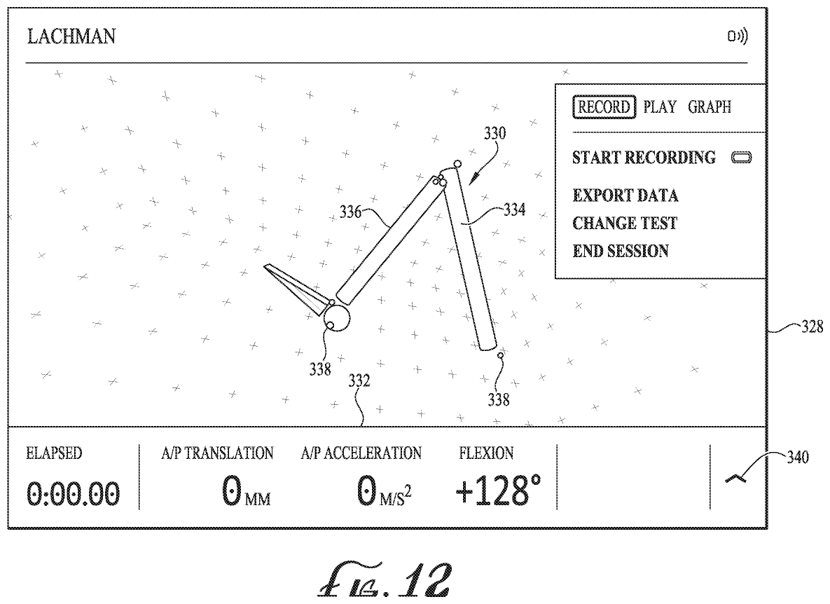

FIGS. 12, 13, and 14 illustrate a display screen showing a limb and its movement according to an exemplary embodiment. After the test is selected, representation screen 328 of FIG. 12 is displayed on the display 104. The representation screen 328 provides a 3D representation 330 of the patient's leg and real-time parameters 332. Included in the 3D representation 330 are the femur 334, tibia 336, and landmark locations 338. Real-time parameters 332 are dependent upon the test selected.

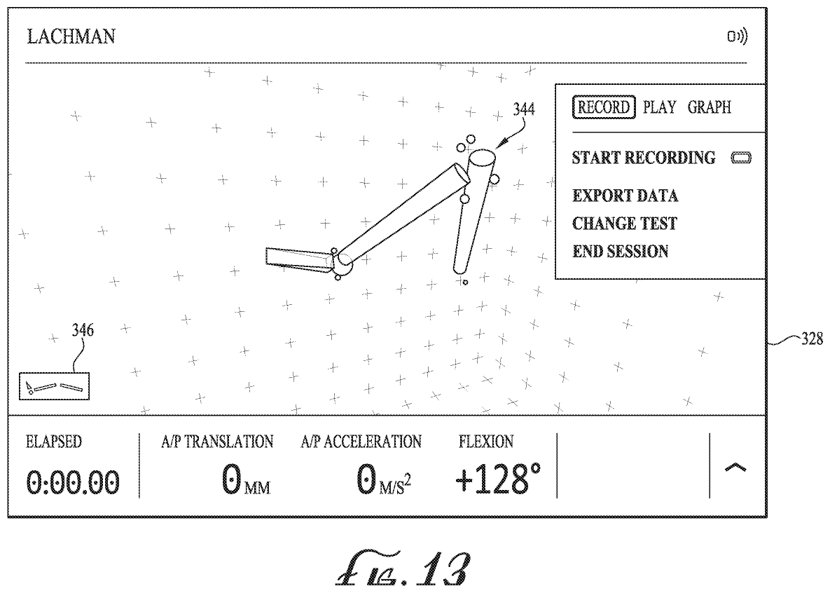

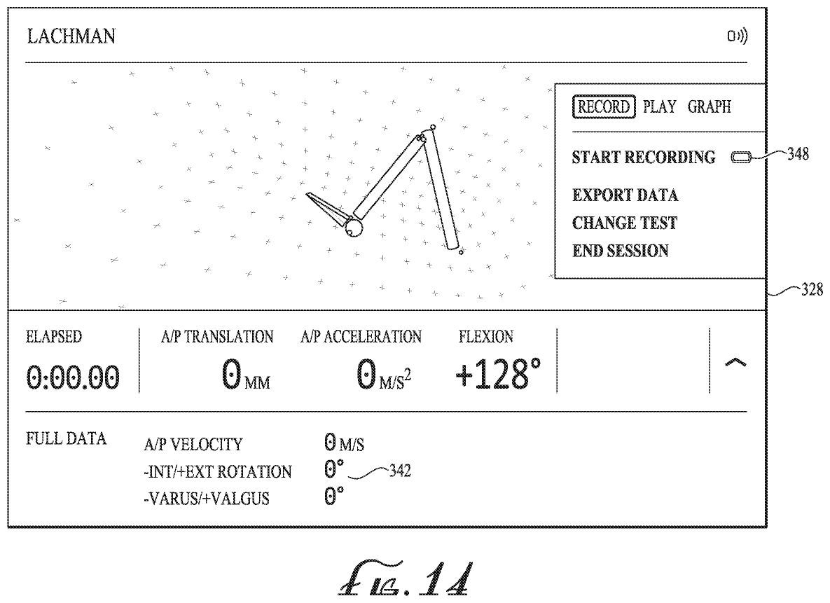

If a user taps on up-chevron 340, test parameters 332 will expand and display additional test parameters 342, as illustrated in FIG. 14. The user can reorient 3D representation 330 by dragging a finger across the 3D representation 330 shown on the touchscreen display 104. When 3D representation 330 is reoriented it becomes a reoriented representation 344, realignment button 346 appears, as illustrated in FIG. 13. When the realignment button 346 is pressed, the processor 102 causes the reoriented representation 344 to return to the original 3D representation 330, as illustrated in FIG. 12.

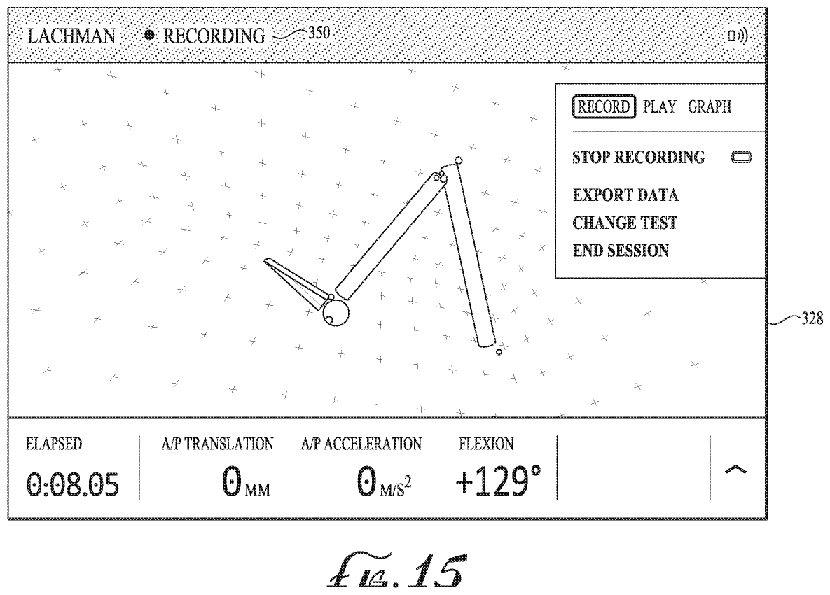

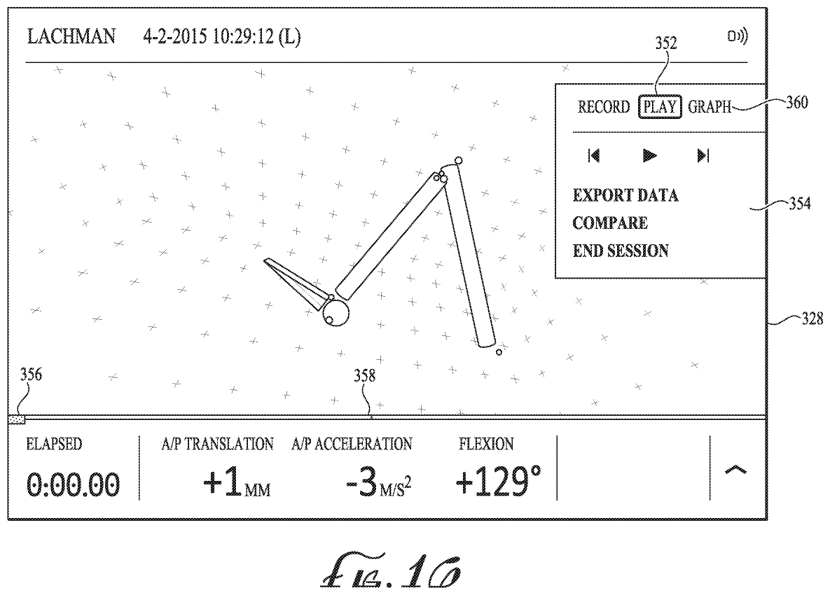

FIGS. 15 and 16 illustrate a display screen showing the recording and playing of a limb movement measured and quantified according to an exemplary embodiment. Recording of measurements can be initiated by using navigation box 348 of FIG. 14 such as by selecting the start recording button using the touchscreen display 102 or a mouse (not shown). Additionally, recording of measurements may be initiated using a footswitch or a button on the stylus 118. Recording indicator 350 appears while the system is recording measurements, as illustrated in FIG. 15. Recording can be stopped by either of the methods used to initiate recording. The user may now choose another test to perform, replay the test, graphically review the results, compare results to previously collected data, or export the data.

When play 352 within navigation box 354 is selected, the application enters play mode, as illustrated in FIG. 16. Play mode allows the user to review the maneuver that was performed. Sweeper 356 may be dragged horizontally to move playback to different points in time. Chapter mark 358 indicates the end of a recording session/beginning of the next recording session. The number of chapter marks 358 depends on the number of discrete recordings made during a test session.

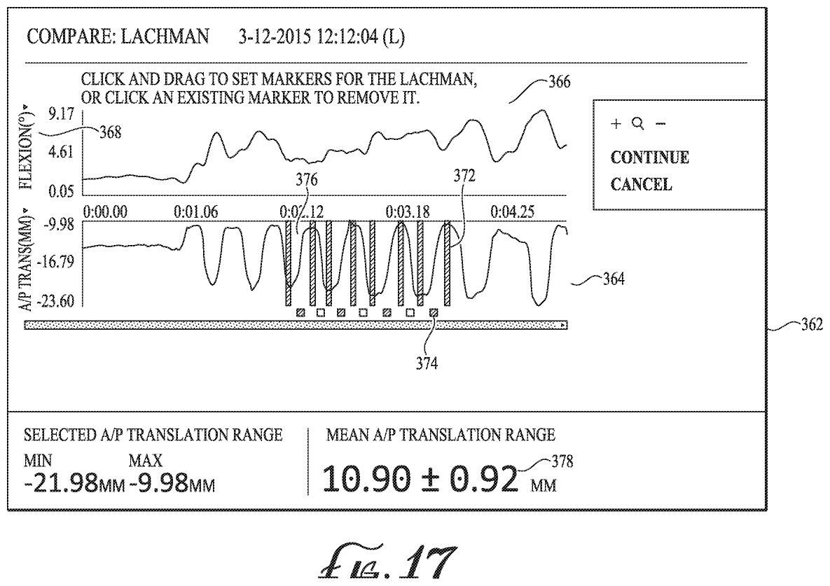

FIG. 17 illustrates a display screen showing a flexion degree and translation plotted against time based on a limb movement measured and quantified according to an exemplary embodiment. When graph 360 within navigation box 354 is selected, a graphing screen 362 is shown on display 104 that graphically displays the test results. Graphing screen 362 shows two graphs 364, 366, which plot the user's desired parameters against time.

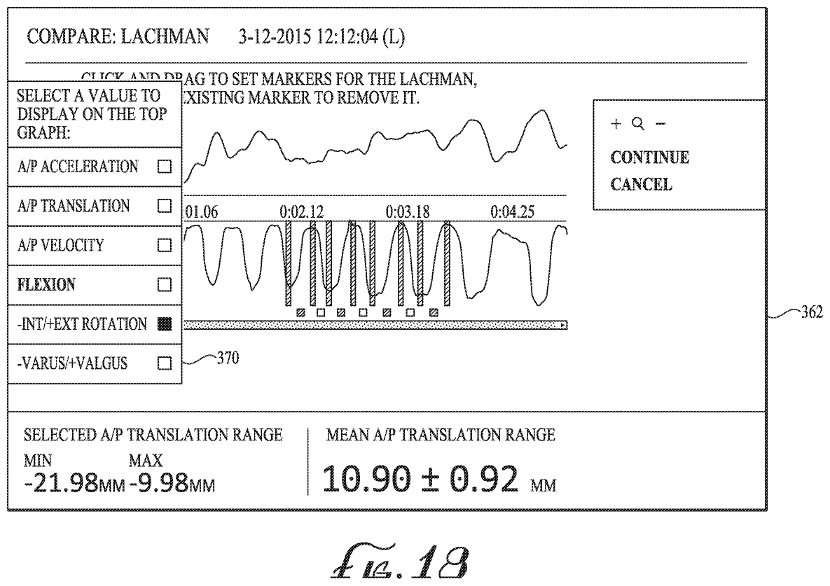

FIG. 18 illustrates a display screen from which a user can select at least one of the acceleration, translation, velocity, flexion degree, internal/external rotation, and varus/valgus information of a limb to be displayed according to an exemplary embodiment. Selecting a y-axis label 368 on either graph of FIG. 17 will allow the user to change the y-axis value for that particular graph by displaying a parameter box 370 as shown in FIG. 18.

When a user selects anywhere within the graphs 364, 366 of graphing screen 362, as shown in FIG. 17, bar 372 will is appear on the graph. When multiple bars are placed within a graph, selection square 374 is placed between each set of bars 372. If a user selects a particular selection square 374, then the area adjacent the selected selection square becomes a shaded inclusion area 376. The peak-to-trough value within the inclusion area 376 is recorded and shown in results region 378. Bars 372 may be repositioned by selecting the desired bar 372 and then dragging. A particular bar 372 may be removed by double selecting it.

According to another embodiment of the present invention, a flexion degree may be plotted on a same graph as a translation. This allows a user to review the translation in conjunction with the flexion degree.

When a Pivot-Shift test is performed on a leg, the translation, i.e., anterior-posterior translation (A/P trans) may be measured in mm. The peaks and lows within the translation can be identified by bars 372. For instance, a first bar 372 may be drawn to overlap with a peak of the A/P trans and a second bar can be drawn to overlap with a bottom of the A/P trans, as illustrated in FIG. 17. The processor of processor 102 can calculate the value of a Pivot-Shift reduction between the bars. For instance, a Pivot-Shift reduction corresponding to the inclusion area 376 may be calculated.

The processor of processor 102 can also calculate the distance between two points of the A/P trans according to the equation below:

PSr=|ΔP|, where ΔP is change in A/P trans from point 1 to point 2

Velocity (PSv) can then be calculated by dividing the PSr with a sampling frequency, according to the equation below:

PSv=|P1−P2|/Hz

Acceleration (PSa) can also be calculated by dividing a different between the calculated velocities by the sampling frequency, according to the equation below. PSa may be one of type of information that is preliminarily shown on display 104.

PSa=|PSv1−PSv2|/Hz

The information provided from these calculations can then be used to populate a data table as shown below. The table can accept any number of sample values.

| Sample # | | | | | |
|---|---|---|---|---|---|
| 1 | 2 | 3 | 4 | 5 | mean ± δ + dV |

Once the table is populated, the information may be stored for later use. For instance, a user may collect similar data from the other leg of the patient for comparison. The data from the two legs can then be compared.

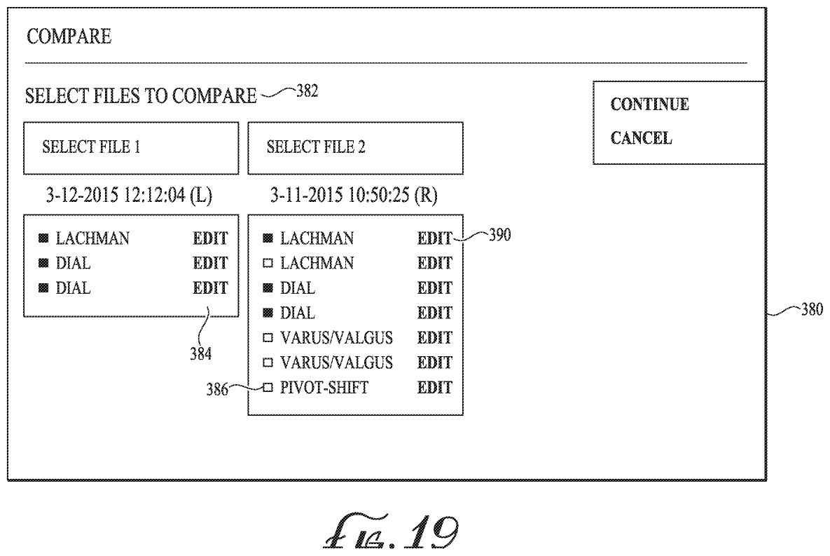

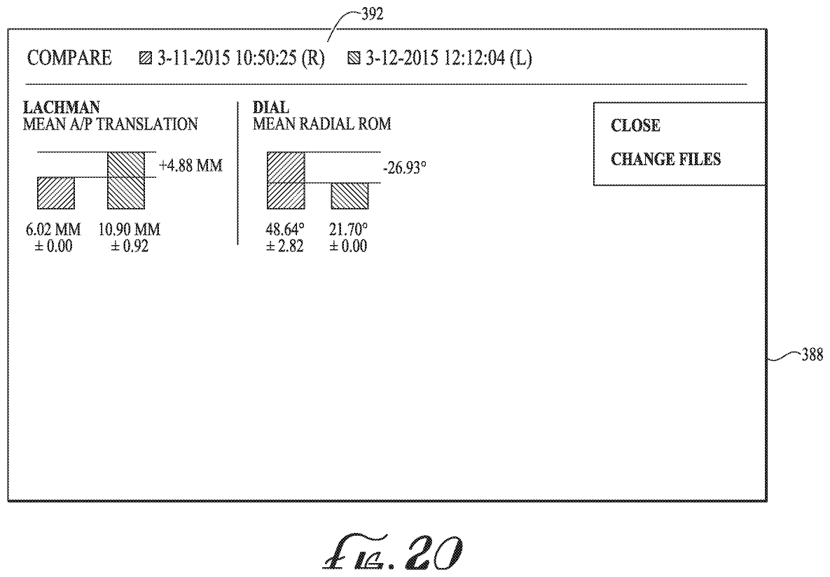

FIGS. 19 and 20 illustrate a display screen from which a user can select stored limb movement information so that the stored information is displayed as a mean of comparison with other limb movement information according to an exemplary embodiment.

When the user selects review/compare mode 306 on start page screen 300 of FIG. 5, file selection screen 380, as shown in FIG. 19, is shown on display 104. The user has the option of selecting one or two separate files for review or comparison, respectively. Selecting the file selector buttons 382 brings up an explorer window for the user to choose the files of interest. The available tests performed within a particular file are shown in the test table 384. Selection squares 386 allow the user to decide which tests will be included within comparison screen 388 shown in FIG. 20. Selecting an edit button 390 besides a test allows the user to modify the inclusion areas 376 reported in the graphing screen 362 of FIG. 17.

The comparison screen 388 of FIG. 20 aligns matching tests from the two files side-by-side and in different colors for visual referencing. The comparison information shown in exemplary FIG. 20 is based on A/P trans, but according to another embodiment of the present invention, velocity and acceleration information of two different legs may also be compared. Selected files are indicated at the top of the screen by their date, time of data capture, and leg side 392.

Embodiments of the present invention are advantageous because they allow for measurements and diagnosis relating to a joint line without measuring at the actual joint line. Embodiments of the present invention avoid the use of cumbersome measuring equipment on a possibly damaged joint and allow for variable placement of sensors around sensitive or damaged areas. Additionally, embodiments of the present invention can capture, display and store detailed data about limb function and allow for comparison between limbs.

Although the present invention has been described in relation to particular embodiments thereof, many other variations and modifications and other uses will become apparent to those skilled in the art. For example, system 100 and process 200 may be applied to other joints, such as the shoulder or ankle, to determine rotational and drawer laxity.

What is claimed is:

1. A system for quantifying joint characteristics, comprising:
an electromagnetic field generator configured to generate an electromagnetic field;
a plurality of electromagnetic sensors positionable inside of the electromagnetic field, wherein each sensor is positionable at a different location on a limb and wherein each sensor is configured to generate position and orientation data;
an electromagnetic stylus positionable inside of the electromagnetic field, wherein the stylus is configured to generate position and orientation data when activated;
a processor coupled to the plurality of electromagnetic sensors and the stylus and configured to receive the data generated by the plurality of sensors and the stylus, wherein the processor is further configured to calculate at least one of an angular movement of the limb and a translation of an appendage coupled to a joint, wherein the limb comprises the appendage and the joint; and
a display coupled to the processor and configured to display at least one of the calculated angular movement and translation.

2. The system of claim 1, wherein the system is configured to further display movement of the limb based on the data generated by the plurality of sensors.

3. The system of claim 1, wherein the plurality of sensors are configured to measure movement of the limb while at least one of a Lachman, Dial, Varus/Valgus, and Pivot-Shift test is conducted on the limb.

4. The system of claim 1, wherein the processor further comprises a memory configured to store at least one of the calculated angular movement and translation per test conducted on the limb.

5. The system of claim 1, wherein at least one of the plurality of sensors is removably attachable to the limb with at least one of a strap, tape and adhesive.

6. The system of claim 1, wherein at least one of the plurality of sensors is removably attachable to a bone of the limb with at least one of a pin, screw and fixture.

7. The system of claim 1, wherein the system is configured to display the calculated angular movement and translation of at least one of a left limb and a right limb.

8. The system of claim 1, wherein the system is configured to simultaneously display the calculated angular movement and translation of left and right limbs.

9. The system of claim 1, wherein the joint comprises a knee joint, and wherein the appendage comprises a femur and a tibia.

10. The system of claim 9, wherein the translation is anterior-posterior translation.

11. The system of claim 9, wherein the system is configured to further display a first vertical line in conjunction with a peak of the displayed translation versus time and a second vertical line in conjunction with a low of the translation displayed versus time, and wherein the translation is calculated during a Pivot-Shift test conducted on the limb.

12. The system of claim 11, wherein the processor is configured to further calculate a Pivot-Shift reduction between the first and second vertical lines.

13. The system of claim 9, wherein the processor is configured to further calculate a distance (PSr) between two peaks of the translation displayed while a Pivot-Shift test is conducted on the limb.

14. The system of claim 13, wherein the processor is configured to further calculate at least one of a velocity (PSv) by dividing the distance (PSr) by a sampling frequency and an acceleration (PSa) by dividing a difference between the calculated velocities by the sampling frequency.

15. The system of claim 14, wherein the system is configured to further display at least one of the velocity (PSv) versus time and acceleration (PSa) versus time.

16. The system of claim 14, wherein the processor further comprises a memory configured to store the calculated velocity and acceleration information per limb on which the Pivot-Shift test is conducted.

17. A method for quantifying joint characteristics comprising:
generating an electromagnetic field around a limb;
placing a plurality of electromagnetic sensors at different locations on the limb;
measuring an initial location of a plurality of landmarks of the limb using an electromagnetic stylus;
measuring movement of the plurality of electromagnetic sensors;
calculating at least one of an angular movement of the limb and a translation of an appendage coupled to a joint using the initial locations of the plurality of landmarks of the limb and measured movement of the plurality of electromagnetic sensors, wherein the limb comprises the appendage and the joint; and
displaying at least one of the calculated angular movement and translation.

18. The method of claim 17, wherein the method further comprises determining a location of the limb using the initial locations of the plurality of landmarks of the limb and measured movement of the plurality of electromagnetic sensors; and displaying an image of the limb based on the determined location.

19. The method of claim 18 further comprising displaying a movement of the limb based on the measured movement of the sensors.

20. The method of claim 17, wherein the step of measuring movement comprises measuring the movement of the limb while at least one of Lachman, Dial, Varus/Valgus, and Pivot-Shift tests are conducted on the limb.

21. The method of claim 20 further comprising: recording the measured movement of the limb during the test; and storing the angular movement and translation information per limb.

22. The method of claim 21 further comprising simultaneously displaying the stored angular movement and translation information of left and right limbs on a same screen.

23. The method of claim 22 further comprising: displaying a first vertical line in conjunction with a peak of the translation displayed versus time and a second vertical line in conjunction with a bottom of the translation displayed versus time, wherein the translation is calculated while a Pivot-Shift test is conducted on the limb.

24. The method of claim 23 further comprising calculating a Pivot-Shift reduction between the two vertical lines.

25. The method of claim 24 further comprising calculating distance (PSr) between two peaks of the translation displayed while a Pivot-Shift test is conducted on the limb.

26. The method of claim 25 further comprising calculating at least one of a velocity (PSv) by dividing the distance (PSr) by a sampling frequency and an acceleration (PSa) by dividing a difference between the calculated velocities by the sampling frequency.

27. The method of claim 26 further comprising displaying at least one of a velocity (PSv) versus time and an acceleration (PSa) versus time.

28. The method of claim 27 further comprising storing the at least one of velocity (PSv) and acceleration (PSa) information per limb on which the Pivot-Shirt test is conducted.

29. The method of claim 17 further comprising: prompting a user to enter a plurality of landmarks of the limb using the stylus; and receiving location and orientation data for the plurality of landmarks of the limb.

30. The method of claim 29 wherein the step of prompting the user to enter a plurality of landmarks further comprises graphically prompting the user to enter location information for at least one of a greater trochanter, medial epicondyle, lateral epicondyle, intersection of the knee joint line and medial collateral ligament, fibula head, medial malleolus and a lateral malleolus.
